(19) United States
(12) Patent Application Publication
(10) Pub. No.: US 2004/0258617 A1
(43) Pub. Date: Dec. 23, 2004

(54) Physical, chemical, and isotopic (atomic) labels

(75) Inventors: Paul J. Weber, Ft. Lauderdale, FL (US); Brian D. Andresen, Livermore, CA (US)

Correspondence Address: John P. Wooldridge, 1334 Ridgestone Court, Livermore, CA, 94551, US

(73) Assignee: Pearl Technology Holdings, LLC.

(21) Appl. No.: 10/463,361

(22) Filed: Jun. 17, 2003

Related U.S. Application Data
(60) Provisional application No. 60/389,707, filed on Jun. 17, 2002.
(60) Provisional application No. 60/389,741, filed on Jun. 17, 2002.

(51) Int. Cl. A61K 49/00

(52) U.S. Cl. 424/9.1

(57) ABSTRACT

Chemical or isotopic labels are added to, e.g., a potentially lethal drug formulation, to generate a unique chemical fingerprint. Combinations of chemical additives are mixed with the drug to aid in their isolation and identification, especially when such drugs are used for illicit purposes. When stable isotopes are incorporated into lethal drugs, the labeling process conveys a very unique internal chemical signature and greatly aids in the identification of the parent drug in body fluids and tissues. When heath-care providers become aware that certain drugs can now be easily tracked and identified in a victim, individuals may be reluctant to utilize these agents for ill purposes.

### BACKGROUND OF THE INVENTION

1. Field of the Invention

The present invention relates to forensic science, and more specifically, it relates to techniques for ascertaining the presence of particular chemicals within a body.

2. Description of Related Art

On Mar. 11, 1998, a respiratory therapist working at the Glendale Adventist Medical Center (GAMC) in southern California confessed to the Glendale police that he had expedited the deaths of 50 patients. He claimed to have overdosed selected patients with either Pavulon (pancuronium bromide) or succinylcholine chloride (SCC) and also to have hastened the death of other patients by other means. The Glendale police subsequently arrested the suspect. However, soon after, he recanted his confession and was released due to a lack of evidence to support police accusations and his confession.

During 1998, a Glendale Police Department task force reviewed the records of thousands of patients who had died at hospitals where the suspect was employed. Following a medical review of questionable deaths, the task force exhumed 20 bodies for toxicology analyses of tissues and fluids to detect the presence (or absence) of pancuronium bromide or succinylcholine chloride. A major focus of the work was to answer a question from the Los Angeles County District Attorney's office: “Were there detectable amounts of either pancuronium bromide or succinylcholine chloride in any of these exhumed tissue samples?”

Succinylcholine chloride, Pavulon, and other similar drugs are skeletal muscle depolarizers. They interfere with nerve transmission and stop muscle contraction. They are used extensively in hospitals and at accident scenes to set broken bones, allow intubation for artificial breathing, and in general surgery. Pavulon is also used for lethal injections in prisoners to carry out death sentences. Inappropriate use of these types of drugs can easily kill an individual through respiratory paralysis. The safety margin is narrow for all these powerful drugs.

A literature review of the two suspect drugs associated with the Glendale investigation first revealed that succinylcholine chloride (SCC) is very unstable and it is metabolized easily. The compound has a very short half-life and is hydrolyzed to two compounds, succimic acid and choline, that are normal constituents in biological tissues and fluids. Thus, prior to the present invention, confirmation of the presence of SCC in autopsy or exhumed tissues was not possible due to its degradation to compounds normal to human (an animal) biochemistry.

In contrast, Pavulon (and other similar drugs) are prepared from starting materials that are not normal to human (or animal) metabolism. However, this drug is highly potent as a skeletal muscle nerve transmission blocker and therefore is given at very small doses (microgram/kg body weight). This makes it exceedingly difficult to detect in biological fluids and tissues (even with the most sophistical and sensitive analysis instruments). The detection of any of these types of drugs is made even more difficult if they are to be isolated and identified in tissues that are aged autopsy samples, retrieved from exhumations.

Careful sample preparation and extensive analytical chemistry investigations finally resulted in the findings that Pavulon was present in six of the 20 exhumed Glendale patients associated with the respiratory therapist. He was subsequently re-arrested and sentenced to 6 consecutive life sentences plus 15 years for attempted murder on another patient. The investigation findings and statements from the respiratory therapist revealed that he had killed well over one hundred patients in hospital settings and if accurate, would establish him as one of the most deadly serial killers in American history.

Other similar (and disheartening) reports have surfaced recently concerning the inappropriate use of pharmaceutical agents in hospital and rest-home settings in the United States (e.g., New York, Illinois, California, Texas, Florida, Ohio) and other countries (e.g., Poland, England, Africa). In these cases, patients were killed because the medical personnel generally considered it impossible to detect the lethal drugs, especially when administered at low dosage amounts in ill patients. During subsequent investigations, the question is always asked of the forensic scientist “Are there detectable levels of the suspect drug (or any unusually potent drugs) present in exhumed tissues samples?” However, depending upon the type of drug, conditions of autopsy or exhumed tissues samples, age of the tissues, and techniques being employed for the analysis, the task of identification of a lethal drug has often been insurmountable. Thus, it is probable that many patients have been killed and others will in the future die due to a feeling by many in the health care field that these drugs cannot be detected easily in their victims.

There is an urgent need, which can be met by this invention, to reduce and/or eliminate the use of lethal drugs in homicides.

### SUMMARY OF THE INVENTION

It is an object of the present invention to reduce and/or eliminate the use of lethal drugs in homicides by chemically or isotopically labeling such drugs to aid in their identification in body fluids and tissues.

An object of the invention is to provide techniques for labeling suspect drugs, such as Pavulon, SCC, Vecuronium bromide, decamethonium bromide, etc., with stable isotopes on the parent drug at positions that do not influence its normal biological response and efficacy.

These and other objects will be apparent based on the disclosure herein.

When heath-care providers become aware that certain drugs can now be easily tracked and identified in a victim, individuals may be reluctant to utilize these agents for ill purposes.

The term “physically labeled” relates to adding other markings on packaging that grab the attention of all that a potentially lethal substance is being used. Physical labeling concerns itself with materials usage. Some examples include: (1) uniquely colored or shaped bottles that contain the lethal drug, (2) highly colored and/or uniquely shaped syringes used to draw up the drug that comes and only interfaces to a uniquely shaped storage bottle, (3) septum tops with unique color or designs, (4) highly visual colored plastic-wear used to dispense the drugs, etc., (5) a unique two-persons dispensing drug-therapy system that will not allow lone heath care personnel to be alone with the patient when administering lethal drugs. (As an aside, all banks follow strict rules that will not allow vaults or the bank itself to be opened by one person). All these physical labels will clearly identify that a potentially lethal drug formulation is being used in a patient. In addition, the completed drug package also includes stringent tracking and accountability capabilities. This can include, but is not limited to electric tagging.

The term “chemically labeled” relates to adding (organic, metallic, and inorganic substances) to a potentially lethal drug formulation to generate a unique chemical fingerprint Often, a potent drug is administered in very low amounts. The carriers and additives in the drug formulations are always present (at the time of administration) at much higher concentrations. For example, in most formulations, benzaldehyde is added at a few percent as both an antioxidant and antibacterial. Additionally, to prepare injectable solutions at the proper pH, various organic and inorganic salts are added to buffer the dosage form. Usually, these buffers are also in high concentration. In order to chemically label potentially lethal drug dosage forms, these additives could themselves be labeled with stable isotopes, unique suites of inorganic or inorganic compounds, or dyes to convey a unique color and chemical signature for the drug preparation. Because these compounds are at much higher concentration, they would be more easily observed in biological fluids and tissues and point to the presence of the lethal drug. One embodiment of this invention includes any combinations of chemical additives, which can be mixed with the drug to also aid in the isolation and identification of lethal drugs used for illicit purposes.

The term “isotopically labeled” relates to incorporating stable isotopes into the parent drug. At reasonable concentrations, stable isotopes are not at all harmful to biological systems. Gram quantities of stable isotopes are in all tissue and body fluids naturally. However, alterations of the natural stable isotope ratios are easily detected with modern analysis instrumentation. When stable isotopes are incorporated into lethal drugs, the labeling process conveys a very unique internal chemical signature and greatly aids in the identification of the parent drug in body fluids and tissues. Isotopes such as carbon-13, deuterium, nitrogen-15, oxygen-18, silicone-29, sulfur-34, etc., can be synthesized into the parent drug (altering its natural stable isotope ratio) in place of the naturally occurring isotopes (e.g., replace selected hydrogen atoms with deuterium). The invention of these new synthetic drugs with stable isotopes would have an immediate impact on their detection at ultralow concentrations in decomposed body fluids and tissues. Mass spectrometry is a key tool to aid in screening samples for stable isotopes. In one example, the parent drug can be prepared by labeling it with just three deuterium atoms in place of three hydrogen atoms. This procedure increases the weight of the parent drug by three mass units. When an equal amount of non-labeled drug is added to the isotoptically labeled signature compound, an artificial isotope pattern of three mass units is observable during any mass spectrometry analysis of body tissues and fluids. The molecular weight region in the mass spectral data would clearly reveal a three mass unit doublet This three mass unit doublet is not natural and is easily seen in complex biological samples, clearly identifying the administered drug.

Virtually, any combination of stable isotopes can be placed into lethal drug preparations to generate a unique chemical signature. As mentioned above, stable isotopes are not harmful to biological systems. As an example, any one or combination of carbon atoms of any of the skeletal depolarization drugs could be replaced with carbon-13 without altering the efficacy of the dosage form. In contrast, the only stable isotope consideration is that when drugs are labeled with deuterium, the newly synthesized drugs do not exhibit diminished or altered efficacy. Normal metabolism of a drug may require site-specific enzyme binding. This binding can be altered if deuterium is in a strategic location on the molecule. When used in patients, the isotopic tags will be transparent to both the physician and patient. Only when circumstances warrant an investigation will these isotopes be looked for in complex biological matrixes.

### BRIEF DESCRIPTION OF THE DRAWINGS

FIG. 1 shows a tamper resistant drug vial dispenser with color indication of lethality.

### DETAILED DESCRIPTION OF THE INVENTION

The invention provides designs for synthetic drugs and modifications of existing drug formulations and toxic chemicals to provide specific labels for their easy identification using physical means and/or chemical or stable isotope tags for use in humans and animals. As a first example, these labeling techniques are most appropriate for skeletal muscle depolarization preparations, but isotopically labeled compounds can be made from other drug modalities (e.g., fentanyl and its analogues, morphine-based drugs, and other abused substances).

Succinylcholine Chloride—As a first specific example, this invention relates to stable isotope labeled succinylcholine chloride (SCC), a powerful skeletal muscle depolarization drug used in surgery and for patient care. The purpose of a stable, isotopically labeled form of the drug will be to enhance its identification at extremely low concentrations in body tissues and fluids. The difficulty in the detection of SCC is due in large part to its short half-life in the blood (minutes) and concomitant metabolism to compounds that are normally in the body, namely choline and succinic acid. If any or parts of this SCC molecule were labeled with a stable isotope, it could be easily identified in very complex biological matrixes. Many combinations of stable isotopes have been formulated for this one drug alone. One example is seen below.

(¹³CH₃)₃N⊕—(CH₂)₂—OH+ClCO—(CH₂)₂—COCl, which is Carbon-13 labeled choline plus Succinylchlroide, combines to form (¹³CH₃)₃N⊕—(CH₂)₂—O—CO—(CH₂)₂—CO—O—(CH₂)₂—⊕N(¹³CH₃)₃, —CI₂, which is Carbon-13 labeled Succylcholine chloride (I).

When labeled with six carbon-13 atoms, the molecular-weight of the labeled drug (I) increases by exactly 6 mass units. When broken down through metabolism, the labeled choline will be clearly identified using gas chromatography-mass spectrometry (GC-MS) analysis-techniques as having 3 additional mass units. This will be a simple marker to indicate that this drug has been administered to the patient

In another embodiment of this invention, the succinylchoride molecule can be labeled with carbon-13 and incorporated into succinylcholine through the following scheme.

(CH₃)₃N⊕—(CH₂)₂—OH+ClCO—(¹³CH₂)₂—COCl, which is Carbon-13 labeled choline combined with Succinylchlroide produces (CH₃)₃N⊕—(CH₂)₂—O—CO—(¹³CH₂)₂—CO—O—(CH₂)₂—⊕N(CH₃)₃. —CI₂, which is Carbon-13 labeled Succylcholine chloride (II).

In this example, the succylcholine hydrolysis product, succinic acid, will have two carbon-13 atoms attached. This addition of two mass units to the labeled succinylcholine (II) will clearly allow its hydrolysis products to be differentiated from normal biological chemicals (naturally occurring succinic acid) in the human metabolism. This will then provide proof that labeled succinylcholine had been given to the patient.

As a further example of multiple stable isotope labeling, carbon-13, deuterium, nitrogen-15, and oxygen-18 could be added (through labeled precursors) to the succylcholine parent drug (III) to provide a very specific labeling tag.

(¹³CD₃)₃¹⁵N⊕—(CH₂)₂—¹⁸OH+ClCO—(CH₂)₂-COCl, which is Carbon-13 labeled choline added to Succinylchlroide, produces (¹³CD₃)₃¹⁵N⊕—(CH₂)₂—¹⁸O—CO—(CH₂)₂—CO—¹⁸O—(CH₂)₂—⊕¹⁵N(¹³CD₃)₃. —Cl₂, which is multiply labeled Succylcholine chloride (III).

Many combinations of stable isotopic labeling can be provided in this molecule. There are a very high number of permutations that can be considered. Any combination of single or multiple isotopes added to succinylcholine chloride (or any of its salts) are within the scope of the present invention.

Thus, through synthetic labeling of a number of succinylcholine chloride drugs (I, II, III→Ω), a number of stable isotopic tags that can be incorporated into the parent drug. Their detection in hydrolysis products and metabolites will provide proof that the parent drug was present in the body fluids of the patient. Any labeled hydrolysis products can be identified by GC-MS and will be easily differentiated from normal biochemical compounds and biological fluids. The use of altered stable isotope ratios in drugs will be a beacon for the analytical toxicologist to find and characterize extremely low levels of the parent drug in patients and exhumation stables.

Also the unique tagging of stable isotopes provides a code that can be utilized to identify manufacturers preparing tagged molecules. This new invention will allow drug manufactures of SCC to label the drug and provide it along with the unlabeled (administered drug) to health care professionals. Knowing that this compound can now be detected in biological fluids and tissues (because it has been stable isotopically labeled before given to a patient) may deter individuals and health care workers from using these powerful drugs to kill patients as well as provide an aid to forensic scientists in discovering that a patient had been murdered.

Stable isotope incorporation into the drug, SCC, is but one example. Many other stable isotope-labeling techniques, incorporating synthetic chemistry with stable isotopes, can be applied to many of the drug preparations used in hospitals, rest home environments, or other venues where drugs are needed and have the potential for abuse. Other examples of skeletal muscle depolarization drugs, competitive neurological blocking agents, chemicals that impact human metabolism, and chemicals that are highly toxic to human and animal biochemical systems can also be labeled with stable isotopes. Some of the toxic compounds are not used clinically today in a health care environment. However, medical research near patients is common and many health care settings where highly toxic compounds (e.g., poisons, venoms, toxins, etc.) may be widely available to health care professionals for medical treatment (e.g., anticancer drugs, anticoagulants). Additionally, isotopically stable drugs could be more easily detection in an Olympic setting where the monitoring of drugs in athletic participants is considered by some to be critical for proper oversight and event judging. Veterinary venues are also important considerations. All horse races are monitored for drug abuse in the race animals. Stable isotopically labeled drugs used either as internal standards for analysis or monitored for illicit performance enhancing drugs are additional examples.

Examples of compounds to be considered for stable isotope labeling are listed below, but are not limited to these general classes of drugs and toxic chemical.

1) Skeletal muscle relaxant and nerve inhibition drugs and chemicals

a) Straight chain and aromatic drug examples

i) Succinylcholine chloride (dibromide and diiodide salts)

ii) Decamethonium (decamethylenehexamethyl diamine dibromide)

iii) Mivaron (Mivacurium)

iv) Metubine (Metocurine diiodine)

v) Flaxedil (Gallamine triethiodide)

vi) Tracrium (atracurium dibesylate)

vii) Mylaxen (Hexafluorenium dibromide)

viii) Alloferin (Alcuronium dichloride)

ix) Fazadinium dibromide

b) Steroid examples of skeletal muscle depolarization drugs

i) Pavulon (pancuronium dibromide)

ii) Norcuron (vincuronium bromide

iii) Zemuron (rocuronium bromide)

c) Piperidine and phylpiperidine analgesic examples

i) Meperidine (demerol)

ii) Diphenoxylate

iii) Loperamide

iv) Fentanyl

v) Alfentanil

vi) Sufentanil

vii) Carfentanil

viii) Remifentanil

d) Opioid based drugs

i) Buprenophine

ii) Butorphanol

iii) Codeine

iv) Drocode (dihydrocodeine)

v) Heroin

vi) Hydrocodone

vii) Hydromorphone

viii) Lalorphine

ix) Levallorphan

x) Levorphanol

xi) Methadone

xii) Morphine

xiii) Nalbuphine

xiv) Nalmefene

xv) Oxycodone

xvi) Oxymorphine

xvii) Pentazocine

xviii) Propoxyphene

2) Natural products that are neuromuscular blocking agent

a) Curare drugs (tubocurarine and β-erythroidine)

b) South American poison-arrow frog poisons

c) Tetrodotoxin (from puffer fish)

d) Saxitoxin (clam poison)

e) Bufotoxin (from toad)

f) Ricin

g) Volkensin

h) Snake venoms and lizard toxins

3) Bacterial and fungal products—botulinum toxin, blue-green agea toxin, red tide toxin, fumonisin

4) Chemical warfare agents:

a) Nerve agent:

i) All G agents—e.g. Sarin [GB], GA, GD, GE, GF, etc.

ii) All V agents - VX, Vx, VG, VM, etc.

b) Blister agents:

i) Lewisite (L-1, 2, or 3)

ii) Mustard (H/D) Cl—CH₂—CH₂—S—CH₂—CH₂—Cl

iii) Dimethyl sulfate (CH₃)₂SO₄

iv) Sesqui-mustard (Q) (Cl—CH₂—CH₂—S—CH₂)₂

v) T(Cl—CH₂—CH₂—S—CH₂CH₂)₂O

vi) Posgene oxime (CX) Cl₂—C═N—OH

vii) Nitrogen mustards (HN-1, 2, or 3)

viii) Ethyl-, methyl-, phenyl-dichloroarsine (ED, MD, PD)

c) Blood agents:

i) Hydrogen cyanide (AC) H—CN

ii) Arsine gas (SA) AsH₃

iii) Cyanogen chloride (DK) Cl—CN

d) Choking Agents

i) Chlorine gas (Cl₂)

ii) Phosgene (CG) (Cl—CO—Cl)

iii) Diphosgene (DP) (Cl—CO—CO—Cl)

e) Vomiting Agents

i) Apomorphine

ii) Ipecac

iii) Adamsite (C₆H₅)₂N—AsHCl (DM)

iv) Diphenylchloroarsine (C₆H₅)₂As—Cl (DA)

v) Diphenylcyanoarsine (C₆H₅)₂As—CN (DC)

vi) Excelsior [CH₂(C₆H₄)₂As]—Cl (EX)

f) Tear Gas Agents

i) Chloroacetophenone C₆H₅—CO—CH₂—Cl (CN, Mace)

ii) Chlorine gas (Cl₂)

iii) Bromobenzylcyanide (C₆H₅)—CHBrCN (BBC)

iv) Chloropicrine Cl₃C—NO₂ (PS)

v) Dichloromethylether (Cl—CH₂)₂O

vi) Miscellaneous agents: Pepper spray (labeled capsaosin)

g) Incapacitating Agents Example

i) Apomorphine (thermally degraded morphine)

ii) 3-Quinuclidinyl benzilate (BZ)

iii) Hallucinogens

(1) Tetrahydrocanabinol

(2) Psilocin (Psilocybin from mushrooms)

(3) Mescaline (from cactus buttons)

(4) Bufotoxin

(5) LSD

(6) PCP (“Angel dust”)

(7) Amphetamines

(a) Methamphtamine

(b) Methenedioxy amphetamines—Ecstasy (MDMA), MDA), etc.

iv) Reserpine (from Rauwolfia)

v) Trichloroacetaldhyde/ethanol (“Mickey Finn”)

vi) Drug examples: Barbiturates, Benzodiazepines, Tricyclics, Anesthetics (e.g., Halothan, cyclobutane, ), etc.

vii) 3,3′,5-Triiodothyronine (produces hyperthermia)

viii) Insulin

ix) Blood thinning compounds (Heprin, coumarins, warfarin, indan-1,3-dione) anisindone, etc.)

5) Miscellaneous agents that impact and intoxicate human metabolism

(Because of concerns centered on international terrorism and homeland defense, stable isotopic tags placed in pesticide and other toxic molecules can provide considerable information concerning the manufacturer of the pesticide for law enforcement when investigating how the pesticides got into the hands of terrorist organization. In case of a terrorist incident these stable isotopic tags can quickly determine which international country last lost control of these substances.)

a) Pesticide examples that can be labeled with stable isotopes

i) Organochloride pesticides

(1) DDT and TDE (DDD)

(2) Methoxychlor®

(3) Ethylan (Perthane®)

(4) Dicofol (Kelthane®)

(5) Chlorobenzilate

(6) Hexachlorocyclohexane (HCH)

(7) Chlordane®

(8) Heptachlor®

(9) Aldrin®

(10) Endosulfan (Thiodan®)

(11) Mirex®

(12) Chlordecone (Kepone®)

(13) Dieldrin®

(14) Endrine®

(15) Taxaphenee and toxaphene toxicant A®

ii) Organophosphates

(1) Malathion®

(2) Tetraethylpyrophosphate (TEPP)

(3) Dimethoate (Cygon®)

(4) Oxydemetonmethyl (Meta Systox-R®)

(5) Dicrotophos (Bidrin®)

(6) Disulfoton (Di-Syston®)

(7) Trichlorfon (Dylox®)

(8) Monocrotophos (Azodrin®)

(9) Dichlorvox (Vapona®)

(10) Mevinphos (Phosdrin®)

(11) Ethyl Parathion

(12) Methyl Parathion

(13) Ronnel (Kortan®)

(14) Cruformate (Rueiene®)

(15) Profenofox (Curacron®)

(16) Sulprofoss (Bolstar®)

(17) Diazinon®

(18) Azinphosmethyl (Guthion®)

(19) Tetrachlorvinphos (Gardona®, Rabbon®)

(20) Isofenphos (Oftanol®, Pryfon®)

(21) Chlorppyrifox (Dursban®, Lorsban®)

(22) Methidathion (Supracide®)

(23) Hosmet (Imidan®)

(24) Pirimiphos-Methyl (Acteltic®)

(25) Tetradifon (Tedion®)

(26) Ovex (Ovotran®)

iii) Organosulfur pesticides

(1) Propargite (Omite®)

(2) Tetrasul (trichlorodiphenylsulfide)

iv) Carbamates

(1) Carbaryl (Sevin®)

(2) Methomyl (Lannate®)

(3) Carbofuran (Furadan®)

(4) Aldicarb (Temik®)

(5) Oxamyl (Vydate®)

(6) Thiodicarb (Larvin®)

(7) Methiocarb (Mesurol®)

(8) Aminocarb

(9) Propoxur (Baygon®)

(10) Bendiocarb (Ficam®, Turcam®)

v) Formmamidine pesticides

(1) Chlordimeform (Galecron®, Fundal®)

(2) Formetanate (Carzol®)

(3) Amitraz (Mitac®, Ovasyn®)

vi) Thiocyanate pesticides

(1) Lethane-384 (2-(2-butoxyethoxy)ethyl thiocyanate)

(2) Thanite® (isobornyl thiocyanoacetate)

vii) Dinitrophenols

(1) Dinitrophenol (ortho-, meta-, and para-isomers)

(2) Dinitrocresol (DNOC)

(3) Dinoseb®

(4) (Binapacryl (Morocide®)

(5) Dinocap (Karathane®)

viii) Organotin pesticides

(1) Fenbutatin-oxide (Vendex®)

(2) Cyhexaten (tricyclohexyl hydroxytin)

ix) Botanically derived pesticides

(1) Nicotine (isolated from tobacco leaf)

(2) Rotenone (from legumes)

x) Acylurea pesticides

(1) Teflubenzuron (Dart®, Nomolt®)

(2) Chlorfluazuron (Atabron®, Jupiter®)

(3) Triflumuron (Alsystin®)

(4) Hexaflumuron (Consult®, Trueno®)

(5) Flufenoxuron (Cascade®)

(6) Flucycloxuron (Andalin®)

(7) Novaluron (GR-572)

xi) Fumigant insecticides

(1) Methyl bromide

(2) Ethylene dibromide

(3) Ethylene dichloride

(4) Hydrogen cyanide

(5) Vampam

(6) Telon II

(7) D-D

(8) Chlorothene

(9) Nemagon (DBCP=dibromochloropropane)

(10) Ethyleneoxide

b) Rodenticides

i) Warfarin

ii) Dicumarol

iii) Coumatetrayl

iv) Coumarfuryl

v) Brodifacoum (Talon®)

vi) Bromadilone (Maki®)

vii) Indone (Pival®)

viii) Diphacinone (Diphacin®)

ix) Clioraphacinone (Rozol®)

x) Bromethalin (Vengeance®)

xi) Strychnine

xii) Red Squill (Schilliroside®)

xiii) Compounds that impact the Krebs cycle and are some of most toxic poisons known to humans

(1) Fluroacetic acid (sodium salt=Cmpound 1080®)

(2) Fluroracetamine (Compound 1081®, Fluorakkii-100®)

xiv) Heavy metal salts (e.g., stable isotopes of As, Pb, Ti, Sn, etc.)

c) Because a certain number of pesticide are extremely hazardous to human health it is important to discussion in more detail examples of specific stable isotope labeling in these compounds.

i) Aldicarb (Temik®)

ii) Cliorethoxyphos (Fortress®)

iii) Disulfonton (Di-Syston®)

iv) Fensulfothion (Dasanit®)

v) Issazophos (Triumph®)

vi) Mevinphos (Phosdrin®)

vii) Parathion

d) Phorate (Thimet®)

e) Terbufox (Counter®)

6) Fuels, fuel oils, solvents and other hydrocarbon systems that can also be labeled and tagged with stable isotopes.

In all these discussions concerning drugs, pesticides and other toxic chemical, a solvent system is usually utilized. Pharmaceutical preparations usually take the form of buffered aqueous solution, alcohol and water mixtures, or solid forms containing a number of other chemical materials. These too can be labeled with stable isotopes. These would then be utilized as a tag indicating that a lethal drug had been administered.

Finally, fuels too can be labeled with stable isotopes to indicate usage, pollution monitoring, and misuse of fuel for criminal or negligent activities. Stable isotopes added as tracer compounds would provide manufacturer identifications and provide a level of monitoring control not available previously. For example deuterium labeled hydrocarbons in unique configurations would be ideal to determine pollution sources (e.g., indicating the owner of leaking underground storage tanks that pollute underground water supplied). Select additives to fuels would also be detected in air samples and thus point to sources of fuel used and manufactures of the fuel.

Stable isotopes can be added to different grades of gasoline, oils, fuel oils, kerosene, and other combustible solvents and fuels. The isotopes to be added can be incorporated into any molecules that contain carbon, hydrogen, sulfur, oxygen, and other nitrogen. Many other stable isotope-labeling activities with fuels can be envisioned.

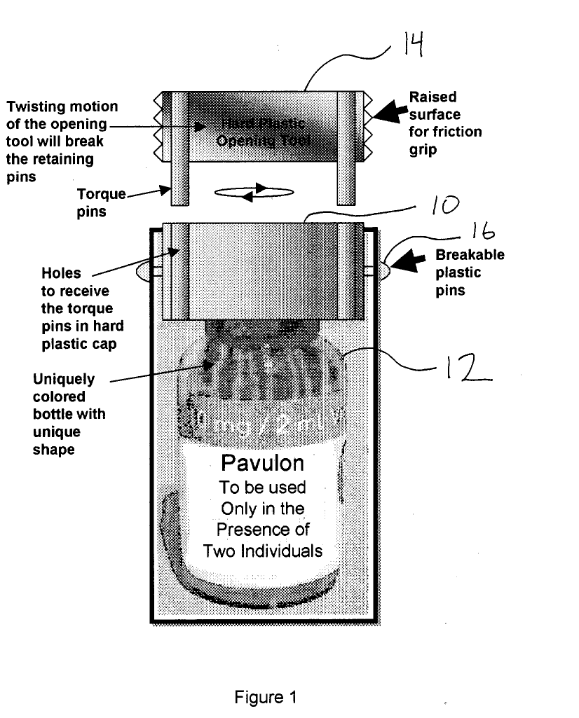

FIG. 1 shows a tamper resistant drug vial dispenser with color indication of lethality. A small hard plastic cap-retainer 10 protects a syringe vial 12 containing lethal drugs. The robust cap-retainer cannot be removed by hand due to the fact that the cap-retainer is flush with the top of the vial packaging. In order to facilitate the removal of the hard plastic cap-retainer, a specialized tool 14 must be used (it may be made accountable and acquired from a locked storage compartment). The cap-retainer opener tool breaks the side plastic retaining pins 16 so that the top can be removed exposing the rubber septum. While one person holds the vial with two hands, another person turns the opener tool to expose the vial septum. The opening is quick, but uses a two-man operation to maintain drug oversight and security.

The foregoing description of the invention has been presented for purposes of illustration and description and is not intended to be exhaustive or to limit the invention to the precise form disclosed. Many modifications and variations are possible in light of the above teaching. The embodiments disclosed were meant only to explain the principles of the invention and its practical application to thereby enable others skilled in the art to best use the invention in various embodiments and with various modifications suited to the particular use contemplated. The scope of the invention is to be defined by the following claims.

We claim:

1. A method comprising labeling a drug with a unique marker to aid in the idenfification of the presence of said drug.

2. The method of claim 1, wherein said drug is located within a biological medium.

3. The method of claim 2, wherein said biological medium is selected from the group consisting of a human or animal body.

4. The method of claim 1, wherein said marker comprises a physical label.

5. The method of claim 1, wherein said marker comprises a chemical label.

6. The method of claim 1, wherein said marker comprises an isotopic label.

7. The method of claim 1, wherein said marker is selected from the group consisting of a physical label, a chemical label and an isotopic label.

8. The method of claim 7, wherein said physical label is selected from the group consisting of (1) a uniquely colored or shaped bottle that contains the drug, (2) highly colored and/or uniquely shaped syringes used to draw up the drug that only interface to a uniquely shaped storage bottle, (3) a septum top with unique color or design, (4) highly visual colored plastic-wear used to dispense the drug (5) a unique two-person dispensing drug-therapy system that will not allow lone heath care personnel to be alone with a patient when administering a drug and (6) a drug package comprising an electric tag.

9. The method of claim 7, wherein said chemical label comprises organic, metallic, or inorganic substances added to a drug formulation to generate a unique chemical fingerprint.

10. The method of claim 7, wherein said isotopic label comprises at least one stable isotope incorporated into a drug to generate a unique isotopic fingerprint.

11. An apparatus, comprising a unique marker added to a drug to aid in the identification of the presence of said drug.

12. The apparatus of claim 11, wherein said marker is readable within a biological medium.

13. The apparatus of claim 12, wherein said biological medium is selected from the group consisting of a human or animal body.

14. The apparatus of claim 10, wherein said marker comprises a physical label.

15. The apparatus of claim 10, wherein said marker comprises a chemical label.

16. The apparatus of claim 10, wherein said marker comprises an isotopic label.

17. The apparatus of claim 10, wherein said marker is selected from the group consisting of a physical label, a chemical label and an isotopic label.

18. The apparatus of claim 17, wherein said physical label is selected from the group consisting of (1) a uniquely colored or shaped bottle that contains the drug, (2) highly colored and/or uniquely shaped syringes used to draw up the drug that only interface to a uniquely shaped storage bottle, (3) a septum top with unique color or design, (4) highly visual colored plastic-wear used to dispense the drug (5) a unique two-person dispensing drug-therapy system that will not allow lone heath care personnel to be alone with a patient when administering a drug and (6) a drug package comprising an electric tag.

19. The apparatus of claim 17, wherein said chemical label comprises organic, metallic, or inorganic substances added to a drug formulation to generate a unique chemical fingerprint.

20. The apparatus of claim 17, wherein said isotopic label comprises at least one stable isotope incorporated into a drug to generate a unique isotopic fingerprint.
